(19) United States
(12) Patent Application Publication
(10) Pub. No.: US 2008/0161730 A1
(43) Pub. Date: Jul. 3, 2008

(54) Method and Apparatus For Chemical Measurement of Sphincters and Narrowing Regions in Hollow Biological Organs

(76) Inventors: Barry McMahon, County Kildare (IE); Hans Gregersen, Hornslet (DK)

Correspondence Address: SUGHRUE MION, PLLC, 2100 PENNSYLVANIA AVENUE, N.W., SUITE 800, WASHINGTON, DC, 20037, US

(21) Appl. No.: 11/816,685

(22) PCT Filed: Feb. 21, 2006
(86) PCT No.: PCT/IE2006/000013

§ 371 Date: Aug. 20, 2007

(30) Foreign Application Priority Data
Feb. 21, 2005 (IE) 2005/0094
Sep. 28, 2005 (IE) 2005/0645

(51) Int. Cl. A61B 5/107

(52) U.S. Cl. 600/593

(57) ABSTRACT

A method and apparatus are disclosed for measuring geometry and compliance in sphincters and other narrowing regions. The apparatus comprises a catheter with an inflatable balloon to be inserted and inflated in the narrowing region. Inside the balloon it is possible to make multiple cross-sectional area or diameter measurements in an axial direction such that a three-dimensional profile of the balloon can be obtained. Preferably multiple sets of electrodes are provided inside the balloon and the cross-sectional recordings are based on measurements of the electrical impedance of an electrically conducting fluid inside the balloon.

### FIELD OF THE INVENTION

The present invention relates to a method and apparatus for making mechanical measurements and derivations in sphincters and narrowing regions in the hollow organs of humans, animals, in such regions in plants and in non-biological systems such as process systems and equipment to evaluate function. The method also relates to an apparatus for performing such compliance tests to sphincters in biological organs and the use of such apparatus.

### BACKGROUND OF THE INVENTION

The function of visceral organs like the gastrointestinal tract, the urinary tract and the blood vessels is to a large degree mechanical. The following introduction refers mainly to the lower oesophageal sphincter (LOS) but the invention relates to similar applications in other sphincters and narrowing regions of hollow organs and of elastic structures in animals, plants and humans. It also has applications in man made machinery and in processes and systems that make use of luminal connections. The phrase narrowing region in the text can be taken to refer to a sphincter or narrowing regions or any intra-luminal area where a geometric change has occurred and it may be helpful to measure it.

The area of the gastrointestinal tract between the oesophagus and the stomach is known as the oesophagogastric junction (OGJ). The mechanism which allows food to pass from the oesophagus into the stomach and controls the amount of food and stomach acids from passing back up into the oesophagus is know as the LOS.

The behaviour of the LOS as a sphincteric mechanism is dependent on two distinct mechanisms. The intrinsic mechanism consists of the internal muscles of the distal oesophagus and the sling fibres of the proximal stomach. The opening in the diaphragm, which provides a path for the oesophagus to pass through, known as the diaphragmatic hiatus, forms the extrinsic mechanism.

The intrinsic muscles are characterized by a resting tension, generally known in a muscle, as tone, which is greater than in the two adjacent segments. The relaxation of the resting tension in the LOS during swallowing allows the passage of food from the oesophagus into the stomach. The function of the LOS can be tested by manometry and indirectly using pH-metry

Manometry is a method of sampling the pressure exerted at various points in the upper gastrointestinal tract. Usually a catheter is inserted either nasally or orally into the oesophagus and pushed all the way down into the stomach. The catheter has either solid state or perfused liquid filled sensors, which can detect the pressure at a certain distance from the oral or nasal opening and in a certain radial orientations.

The most common method of measurement is to pull through the catheter either with a constant pulling or in a stepped fashion. As the pressure sensors reach the LOS in a normal subject there will be a pressure increase indicating the tensioned area of the LOS. Indeed it is common to refer to this area as the high-pressure zone. These pressure measurements are used to determine the peak pressure of the sphincter and overall length of the high-pressure zone.

For pH testing normally the manometry peak pressure finding is used to locate the point 5 cms above the LOS where the pH probe will be located. From there the probe is connected to a monitoring device usually strapped to the patients waist and an accurate record of the patients pH in the oesophagus over a 24 hr period is kept. The most important measure from this test is the amount of time the pH in the oesophagus is <4 indicating that the content in the oesophagus is acidic, suggesting that the oesophagus is exposed to stomach acids which are refluxing into it. Postprandially most people will have acid travel up into the oesophagus but it will usually be for a limited time and this is considered normal. Only when this acid exposure is prolonged is it associated with reflux disease.

Manometry is used to record the activity of the LOS patterns but this gives no information about the geometry and passive mechanical properties and only indirect data on the active mechanical forces and tension in the sphincter.

Although manometry and pH-metry methods provide important data on the acid refluxing and motor function of the LOS, little attention has been paid to biomechanical parameters such as tension and strain and the relationship between biomechanical properties, compliance and sensation. During the past two decades, impedance planimetry has been used in gastroenterology to determine wall tension and strain in animal experiments and human studies. Impedance planimetry provides a measure of cross-sectional area in the LOS and is therefore a better basis than manometry measurement for determination of compliance in the sphincters of the GI tract.

Dysfunction of the LOS can generally be related to two diseased states. Achalasia which is an uncommon primary oesophageal motor disorder that is characterized by incomplete relaxation of the LOS on swallowing and an absence of peristalsis of the oesophageal body, and the much more common occurrence of gastro-oesophageal reflux disease (GORD). GORD can occur when there is over exposure of the oesophagus to acids refluxing back into the oesophagus from the stomach. People suffering from GORD usually have heartburn and may have regurgitation and dysphagia. Recent figures indicate that up to 44% of the U.S. population suffer from GORD. GORD can result in damage to the mucosal lining of the oesophagus, commonly referred to as esophagitis. Of the patients who have chronic GORD, 10-15% may develop a condition called Barrett's epithelium. Patients with Barrett's epithelium have up to a 40 times greater chance of developing a malignancy. Although the underlying cause of GORD is not exactly known it is related to two main patterns of sphincter dysfunction; an abnormally high rate of reflux episodes during transient LOS relaxations and defective basal LOS pressure.

Patients will normally undergo an upper gastrointestinal endoscopy as a diagnosis for GORD which will establish if there is an inflammation of the LOS caused by over exposure of the oesophagus to stomach acids or quite often will identify if there is a hiatus hernia. A hiatus hernia occurs when part of the stomach makes its way through the hiatal opening in the diaphragm and there is a bulging of part of the stomach into the thoracic cavity.

There are numerous treatments for GORD. Of course there are dietary issues such as avoiding large-volume high-fat diets as well as the other foods mentioned early with regard to precipitating factors and avoiding these is key. Fatty foods are known to lower LOS tone, increase the number of TLOSRs, delay gastric emptying and increase oesophageal sensitivity to acid. Weight loss is encouraged in over-weight individuals, which is associated with reducing intra-abdominal pressure. Exercising after heavy meals is to be avoided and stress reduction methods are recommended. It is thought that the role of body position is important. Different patients display different patterns of reflux. Some patients have more reflux when that are lying down and some patients have more standing up or sitting. It is thought that nocturnal or lying down reflux is the most injurious. Recommendations are, remaining upright after meals, raising the head of the bed by 6 inches and lying on the left side in bed.

Over the counter type therapies for GORD such as antacids and half-prescription-strength histamine H₂-receptor antagonists (H₂ blockers) are useful for occasional, as needed therapy. Antacids have a relatively short duration of action (2 to 3 hours). H₂ blockers have a longer duration but slower onset of action. Combination antacid and H₂ blockers medication is now available over the counter but all these medications are only useful for intermittent symptoms of GORD.

Full prescription strength H₂ blockers are useful for treating milder forms of GORD but even at double dose therapy this medication is not as effective as proton pump inhibitor therapy (PPI). PPI's have become the corner stone of therapy because of their ability to inhibit gastric acid secretion. A single dose of a PPI can keep gastric pH above 4 for 50% of the day. This medication has been shown to be safe in short and long term use. This drug has become the most commonly prescribed medication in the world. One pitfall of PPI's is their apparent inability to control nocturnal reflux. However the clinical significance of this is still being debated.

The surgical operation, which is widely used today, is the laparoscopic Nissen fundoplication. In 10 and 20-year studies this operation has shown a 900% success rate. The principle of a Nissen fundoplication is a wrapping of the proximal stomach around the distal oesophagus and LOS. Minimally invasive surgical techniques developed over the last 15 years have made this operation very popular and attractive to many long-term GORD sufferers. It is recommended that these procedures be carried out in centres with the correct surgical expertise, as there is quite a long learning curve for the procedure.

In the last 3 to 4 years a number of newer therapies are emerging for GORD. These include a number of endoscopic suturing devices whose objective is to place stitches and clips in the area of the LOS.

A trans-oral catheter system that delivers radiofrequency energy to the LOS with the idea of fibrosing the area, inhibiting TLOSRs and injection of the LOS with substances that appear also to fibrose or expand the thickness of the LOS wall is also in use.

Another technique showing promise is the idea of injecting or placing an inert substance submucosally or intramuscularly using a special needle assembly. So far patients treated with these therapies have been limited to patients with Non-Erosive Reflux Disease (NERD) and do not have a hiatal hernia of any significant size. These therapies are popular with young people who do not want a life time of PPI therapy.

Results from trials of some of these therapies have not reached the publishing stage but preliminary results do show some efficacy with regard to symptoms. However ambulatory pH data does not always indicate that there has been an improvement in oesophageal acid exposure and suggesting that some of the effect may be due to visceral sensory inhibition.

It is now widely accepted, even by the critics that endoluminal therapies will have a place in future GORD treatment, however all the research to date has been heavily criticised for the lack of control groups and randomised sham trials.

It has also been suggested that pH data may not be the best method of interpreting the success of the procedures.

### SUMMARY OF THE INVENTION

The object of the present invention, according to a first aspect, is to provide a method and an apparatus capable of applying stimuli to sphincters in the GI tract and to simultaneously measure physical properties of the apparatus applying the stimuli, which are representative of the response of the stimulated sphincter. A further object of the invention, according to the second aspect, is to provide a method and an apparatus for simultaneously stimulating and measuring dynamic and static luminal geometric data from humans or animals.

The invention is designed to measure active and passive narrowing regions and their mechanical reactions towards stimulation by introducing a catheter from an exteriorly accessible opening of a bodily hollow system in an animal, human or man made mechanical system or process. This catheter is provided with a bag-like balloon, which from now on will be referred to as the balloon unless otherwise stated, situated between a proximal end and a distal end. The balloon is inflated in the narrowing region and even extending to both sides of the narrowing region which is maximally contracted or some intermediate state obtained during use of pharmacological substances intended for activation or relaxation of muscles or for the influence of nerve functions or during conditions where the narrowing region has been modified by therapy or trauma. Inside the balloon it is possible to make multiple cross-sectional area or diameter measurements in an axial direction such that a three-dimensional profile of the balloon can be obtained and analysed with respect to geometry and mechanical parameters.

The invention comprises a further development of balloon distension methods by providing compliance and distensibility diagrams in vivo before and during the administration of muscle relaxants muscle stimulating drugs or the presence of a physical stimulation. Hereby, active and passive properties can be studied in vivo or in whole segments in vitro and can be related to other physiological responses such as relaxation of the sphincter elicited by the mechanical stimulation.

Development of balloon distension protocols is useful in order to correlate biomechanics, motor control, sensation and the valvular action of the LOS, in particular in the gastrointestinal tract in vivo and in vitro. The distension can be used to derive pressure-cross sectional area (CSA) and pressure-diameter diagrams with subsequent evaluation of the circumferential wall tension, force of closure, strain, compliance and flow characteristics. In relation to the wall tension, it is the active, passive and total forces inducing tension and stress, which is derived. This length-tension test provides data on the passive nature of the tissue, on the maximum force generated by the smooth muscle, and the strain corresponding to the maximum force. These stimulation and measurement conditions may be induced by any combination of pressure volume, impedance planimetry, diagnostic ultrasound or any other developed or developing imaging modality.

The preferred method of cross-sectional measurements is by impedance planimetry either by using multiple sets of recording electrodes on the catheter or by using an oversized bag where the catheter can be moved within the bag, providing a pull-through procedure using fewer sets of electrodes with continuous cross-sectional measurements during the movement in an axial direction.

The object according to the invention is obtained by a method comprising the step of
measuring a change of a physical property of a balloon during inflation of the balloon, said physical property being a dimensional state of the balloon itself, alternatively said physical property being correlated to a force induced to the balloon, alternatively said physical property being a mutual state between the balloon and the narrowing region under investigation, and from the measurement deriving data on compliance of the narrowing region under investigation in order to obtain knowledge on active and passive tissue properties.

If the measurements are performed in vivo, alternatively performed in vitro, the method comprises the initial step of introducing from an exteriorly accessible opening of a bodily hollow system a catheter into the narrowing region under investigation, said catheter being provided with an inflatable balloon situated between a proximal end and a distal end of the catheter.

It is important to notice that by the denomination “balloon” is meant only a bag capable of being inflated. The inflation need not result in a stretching of the material of the balloon. Thus, perhaps the balloon is made of a material, which subsequent to inflation is not subjected to any stretching, but merely has an increased volume due to the inflation. Accordingly, in the whole of the application, apart from in the test results in the last part of the specification, the denomination “balloon” will be used, because this is the commonly used denomination, although the balloon may be as a bag, i.e. no stretching of the bag. However the balloon make be of such a material as PME where upon the balloon initially does display significant elastic properties upon inflation but that once deflation occurs the balloon holds it shape and distended size and in effect obtains bag like properties. This will enable the use of a catheter that has slim and smooth properties so as to minimise the discomfort to patients when it is inserted into any of the body openings where hollow organs can be accessed, all the while ensuring that the balloon contained within it can be re-inflated for the purpose of stimulation and measurement with the minimum amount of error being introduced by the mechanical properties of the balloon being inflated in the region of interest.

In a possible other aspect, a method is claimed for investigating multiple simultaneous measurements of geometry, including surface geometry, competence and distensibility properties in sphincters and other narrowing regions on the human body, animals, plants and non-biological systems, said method comprising
a. placing a balloon in the narrowing region, inflating the balloon as a stepwise distension or a ramp distension with controlled distension rates until the balloon abuts the inner wall of the sphincter in order for the balloon to be fixed longitudinally in relation to the hollow system and for mechanical stimulation of the system,
b. measuring a change of a physical state of the hollow system during inflation of the balloon, said physical state of the hollow system being correlated to a corresponding physical property of the balloon, alternatively said physical state of the hollow system being correlated to a mutual state between the balloon and the wall of the hollow system, and
c. from the multiple measurements of geometric parameters optionally combining such measurements with measured forces, to derive force-deformation properties such as compliance, distensibility parameters, length-tension diagrams, tension-strain diagrams, and stress-strain diagrams under various conditions such as during administration of drugs in animals or persons with diseases.

An apparatus for performing the method according to any aspect of the invention comprises a catheter being provided with an inflatable balloon situated between a proximal end and a distal end of the catheter, and the apparatus comprising means for passing an inflating fluid, preferably a liquid, from the proximal end to the balloon, and furthermore may comprise means for recording and controlling the temperature in the balloon of the catheter.

The geometry of the region of interest may be obtained either in vivo or from a lumen within the measurement probe by using a visualising, ionising radiation or non-ionising radiation medical imaging system, said system being capable of measuring lumen diameters, multiple cross-sectional areas along the length of the balloon, circumferences, the contour, the three-dimensional geometry or the wall thickness of the region of interest with or without the balloon pressure.

The object according to an aspect of the invention is obtained by a method comprising the steps of:
inflating the balloon as a stepwise distension or a ramp distension with controlled distension rates when the balloon is located in the narrowing region under investigation for mechanical stimulation of the system,
measuring a change of a physical state of the narrowing region under investigation during inflation of the balloon, said physical state of the narrowing region being correlated to a corresponding physical property of the balloon, alternatively said physical state of the narrowing region being correlated to a mutual state between the balloon and the narrowing region under investigation, and
from the measurements deriving force-deformation diagrams such as length-tension diagrams, pressure-CSA diagrams, pressure-diameter diagrams, tension-strain diagrams, stress-strain diagrams and pressure-geometry data such as wall thickness, lumen size and safe under various conditions such as during administration of drugs in animals or persons with diseases.

In the case that the measurements are made in vivo, preferably the balloon is introduced from an exteriorly accessible opening of a bodily hollow system a catheter into the narrowing region, said catheter being provided with an inflatable balloon situated between a proximal end and a distal end of the catheter,

An apparatus for performing the method according to any aspect of the invention, a catheter being provided with an inflatable balloon situated between a proximal end and a distal end of the catheter, and the apparatus comprising means for passing an inflating fluid, preferably a liquid, from the proximal end to the balloon, and where the apparatus is provided with means for measuring at least one of the following physical properties of the balloon: the volume of the balloon, the cross-sectional area of the balloon seen in a direction parallel to a longitudinal extension of the bodily hollow system, when the apparatus is introduced into the body, the diameter of the balloon in a plane perpendicular to a longitudinal extension of the bodily hollow system, when the apparatus is introduced into the body, the tension of the balloon, the strain of the balloon, the pressure of a fluid inside the balloon, and the temperature of a fluid inside the balloon, the apparatus intended for measuring a physical reaction of a person or an animal, when a bodily sphincter of the person or the animal is being subjected to a number of artificially applied stimuli.

The apparatus, in a preferred embodiment using impedance planimetry can be used to describe the geometric and mechanical characteristics of luminal areas such as sphincters, valvular regions or other narrowing regions. This requires that the apparatus should be small in diameter and technical construction such be suitable for miniature and sub-miniature embodiments. Geometric and force data will be gathered from electrodes. Electrodes will be in arrays normally but not always in arranged rows along the longitudinal length of the catheter. Electrode construction will be of a conducting material without any insulating surface at the position where the measurement is to take place. This exposed area may be circular or pointed. The order or arrangement in which an electrical signal is detected and measured from any pair of electrodes may be varied and some electrodes may be common to a number of different measurement arrangements.

Electronic hardware and software used to interpret, display, make calculations from and store data, will be configured to allow for different data collection configurations from the probe electrode array. Where suitable, signal multiplexing, synchronised array excitation and data sensing will be used to optimise the use of electrodes and signal lines. Electronic and physical switching may be used to switch between electrode arrays or segments of electrode arrays. Signal analysis from the apparatus may include reconstruction of three dimensional geometry. Sinusoidal wave patterns may be analysed in the frequency and vector domains to help determine transit points from cyclic data such as respiratory movement or cardiac beat movement. This could be used for example in the oesophagogastric junction to determine the point of respiratory inversion as the junction moves from the thoracic cavity into the abdominal cavity in the body.

The geometry of the sphincter or other narrowing region may be obtained by using a multi-electrode impedance planimetry system, said system being capable of measuring multiple cross-sectional areas along the length of the balloon with or without the balloon pressure. Possibly, the signals from the multiple electrodes are handled using electronic multiplexing to minimize the number of electrodes and signal processing channels.

Ring electrodes or point electrodes may be used to detect conductance of electricity in a saline solution as a method of measurement from an impedance planimetry version of the apparatus. Interconnecting wires connecting electrodes at the distal end of a probe assembly to hardware apparatus at the proximal end may be placed inside a lumen or lumens in the catheter of the probe, where miniature wires are run outside the probe or embedded in the wall material of the probe or the interconnections are made via a suitably constructed flexible circuit assembly.

The signals being measured from multiple electrodes may be mounted on a catheter in a probe connected to configured hardware and software sensing signals collected from different connections to the sensing electrodes, thereby optimising the use of the insulated wires running down the lumen and therefore maximising the number of measurements being made.

The apparatus may be an apparatus using an electronic device to provide unique identification the probe and the signal conditioning system. Alternatively or additionally, the apparatus may be using an electronic device to provide unique calibration factors for the probe. Alternatively or additionally, the apparatus may be using an electronic device to code a specific probe with a particular configuration of the signal conditioning system for the purpose of safety. Alternatively or additionally, the apparatus may be using an electronic device to synchronize the transfer of multiplexed signals to and from the signal conditioning system.

Stresses applied are derived from multiple geometric, force and other physical measurements. Also, regional distribution in layer thickness can be measured or derived. Furthermore, sphincter characteristics can be modelled using multilayer models.

Ultimately the objective of any apparatus will be to establish the competence of the sphincteric or narrowing region under investigation. This may be defined as a measure of the distensibility of the region under investigation. It may be represented as in FIG. 2 as a description of the geometric status of the region or changes in the geometric status under pre-defined conditions.

Ultimately in a sphincteric or narrowing region which for example might act as a valve mechanism, then the lowest cross-sectional area (CSA) measured at a certain pressure may be the important parameter. Other factors during measurement may also be critical such as the posture of the subject, any controlled or uncontrolled stimulation of the region of investigation. The temperature of the investigation. The temperature at which the device was calibrated can also be important.

### BRIEF DESCRIPTION OF THE DRAWINGS

The invention will now be described in detail with reference to the drawing, where

FIG. 1 shows a simple sketch of the probe in the oesophagho-gastric junction as an example of an application of the apparatus

FIG. 2 shows a reconstruction of the surface geometry of a sphincter in the body. The data is reconstructed from real CSA data collected in a human subject. The reconstruction assumes that the geometry is cylindrical.

FIG. 3 is a sketch showing an example of the probe according to the invention being used for human sphincters and narrowing regions, for physical stimulation and/or measuring properties.

FIG. 4 shows how the bag-like balloon maintains its approximate position while the position of the electrodes can be changed either left or right of their original position in a longitudinal direction with reference to the lumen.

FIG. 5 shows a schematic for the electronic connections and amplification for an example of the probe. The arrangement is important to optimise the number of measurements from the limited spacing available in the catheter for signal connections.

FIG. 6. shows a close-up of the electrode connections indicating how one connection is made to the second electrode in the lead pair and the first electrode in the trailing pair of electrodes.

FIG. 7 shows placement of an example apparatus placed in a sphincter or narrowing region for the purpose of making geometric measurements.

FIG. 8 is an example of actual data obtained in and around the LOS with an 8 electrode probe system.

### DETAILED DESCRIPTION OF THE INVENTION

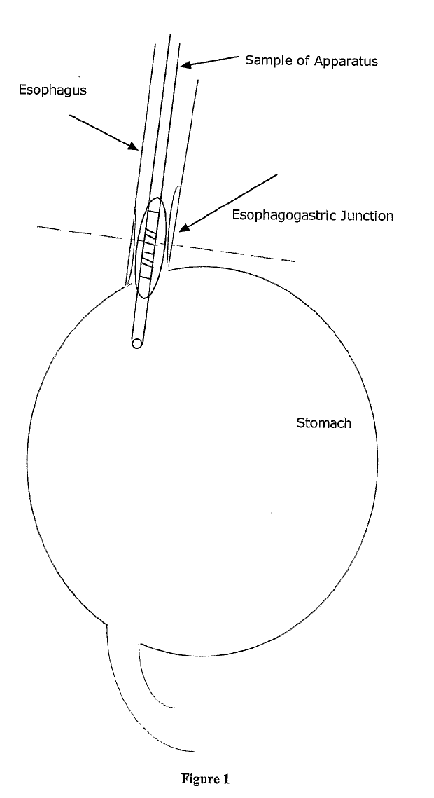

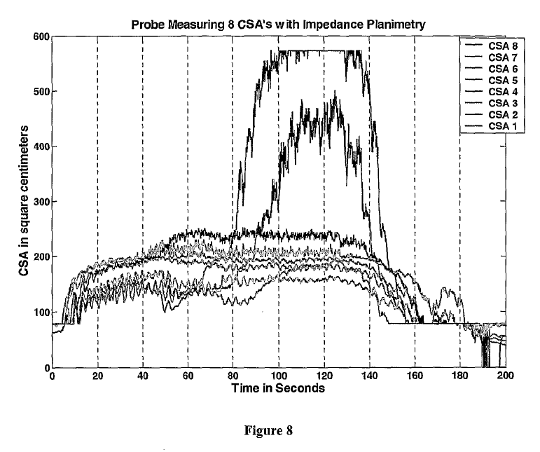

FIG. 1 demonstrates an embodiment of the apparatus for providing stimuli and/or for measuring certain properties in a sphincter or narrowing region in the body. The apparatus consists of a catheter, alternatively denoted a probe, having a proximal end and a distal end. The distal end is introduced into a readily accessible opening from the outside. An example would be its use in the OGJ where it would be placed in the mouth or the nose and pushed further down into oesophagus where it is placed in the OGJ. The probe as positioned in the OGJ and when inflated may appear as in FIG. 7. Placement of the probe in the LOS is as per the already well established technique of oesophageal manometry. Near the distal end, a bag-like balloon is placed. This bag-like balloon is normally made with a non-compliant material so that it doesn't contribute to the resistance to inflation during distension and it is usually large and in a cylindrical or spherical shape. All references to balloon here means a balloon or a bag-like balloon as just described.

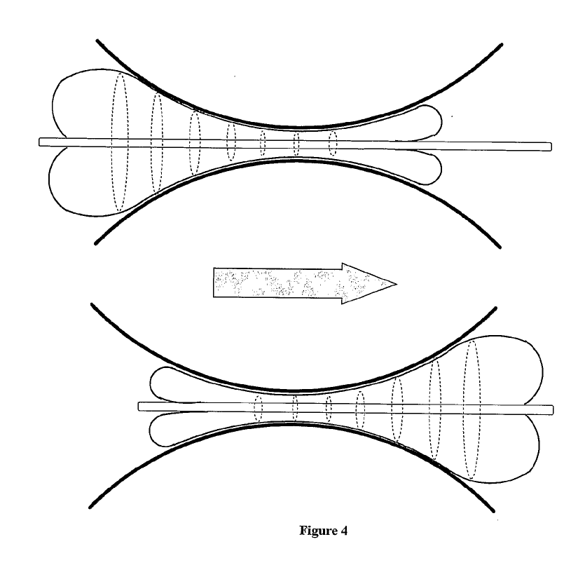

The balloon is inflated so that its outer surface makes contact with the mucosal lining of the oesophagus in the area of the LOS. The inflation takes place only after the catheter has been introduced into the sphincter. By inflating the balloon, the balloon is fixed in relation to the LOS but the catheter is free to be displaced longitudinally along the length of the EGJ but only under control of the operator by adjusting the proximal end of the catheter where it exits the body i.e. at the nose or the mouth as demonstrated in FIG. 4. A rolling displacement of the balloon on the catheter will allow free movement of the catheter longitudinally within a limited distance set by the size of the balloon. This design will enable a pull through or a push through technique to be employed for the catheter. It will also enable some fine tuning of the placement position of the probe to improve measurements.

While the balloon is inflated data on pressure inside the balloon, pressure at the distal end of the catheter, pressure at the proximal end of the catheter and multiple cross sectional areas (CSAs) inside the balloon may be recorded on a personal computer and then they can be used to construct a 3 dimensional geometric surface profile of the sphincter.

For certain measurements the catheter will be fixed at the proximal end as well, i.e. is fixed to the nose or any other outer surface or organ. In the situation shown, where the catheter is introduced through the nose, fixation of the proximal end may take place in any suitable manner, perhaps by a clamp being clamped to the wing of the nose, to the nasal bone or to the bridge of the nose. Once introduced into the bodily sphincter, the apparatus may be used for stimulating the sphincter of a person or an animal by mechanical stimulus. Alternatively, or in addition, the apparatus may be used for measuring a physical reaction of a person or an animal, when the bodily hollow system of the person or the animal is being subjected to a mechanical stimulus of the above-mentioned type.

For certain measurements the catheter may have a second balloon mounted at a fixed distance proximal or distal to the first balloon where this second balloon's internal volume, pressure or CSA may be changed in a controllable and measurable manner as a method of luminal stimulation.

The balloon of the catheter is often made from polyurethane being a material readily extendable, being non-harmful to the human or animal body and having distinct physical properties such as elasticity, modus of strain etc. The thickness of the material, which the balloon is made of, is perhaps between 30 μm and 50 μm. However, any other well-suited material may be used as long as it fulfils the need for enlargement, non-permeability of the fluid inflating of the balloon, perhaps non-permeability of electrical currents, and security towards not being harmful to the body. Thus, polypropylene (PP) or polyethylene (PE) may alternatively be used. Even other materials may be used referring to the fact that the denomination “balloon” need not imply that the material from which the balloon is made is dilated, when the balloon is inflated. Thus, materials having much less elasticity than the above-mentioned materials may be used, perhaps materials which, when inflated, do not show any noticeably elastic deformation. Also the thickness of the material, which the balloon is made of, may be thicker than the dimensions mentioned above, thus perhaps also resulting in that the material, when inflated, does not show any noticeably elastic deformation.

Other materials such as PME will be used whose characteristics are such that they will readily expand to a maximum size needed but upon deflation their elastic properties disappear and they behave bag like. The purpose of using this material will be to ensure minimum discomfort for the patient when the probe is being inserted into the appropriate hollow organ e.g. the nasal cavity or the mouth.

The size of the balloon, when inflated, differs depending on which bodily hollow system the apparatus is used in and depending on which part of the system in question, that the apparatus is used in. The balloon distension may be pressure, volume, CSA, tension and even strain-controlled using stepwise or ramp distension protocols. As an example, for use in the LOS, the balloon may be inflated to a diameter of 25 mm seen in a plane perpendicular to the longitudinal direction of the oesophagus. Some sphincters in the body may extend to smaller or larger cross-sectional areas than the LOS. Thus, the urogenital system will have much smaller sphincters, whereas the anal sphincter will have larger diameters than the LOS, perhaps necessitating a balloon having up to 50 mm in diameter when inflated.

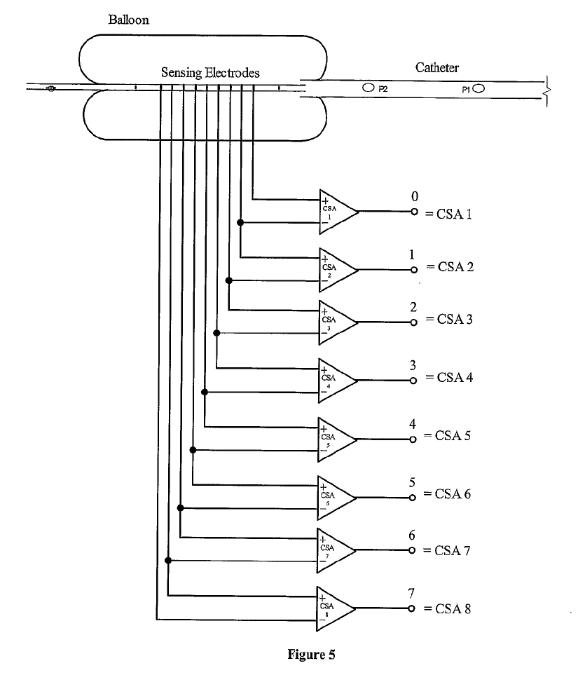

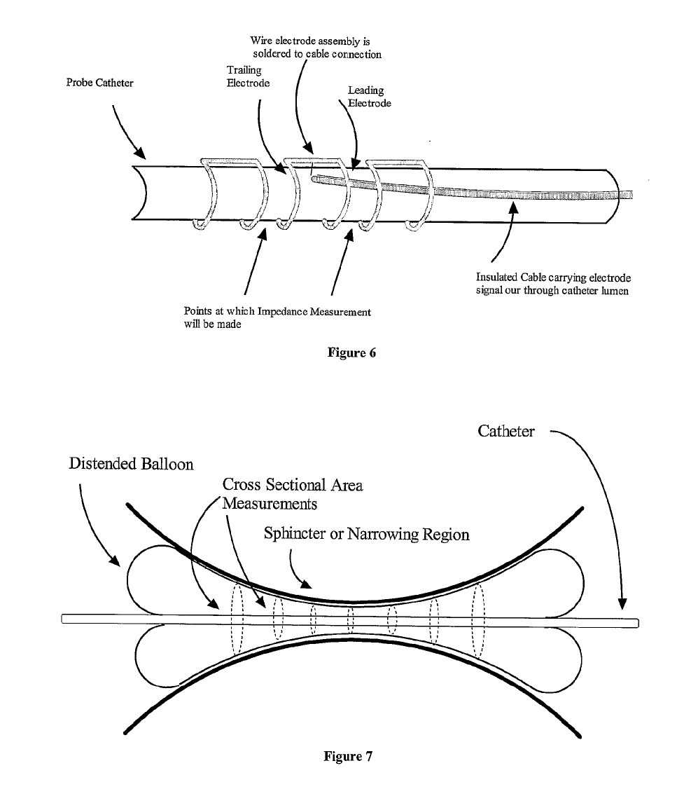

In FIG. 5 a schematic showing the specific connections and signal amplification arrangements for an impedance planimetry embodiment of the apparatus is illustrated. This embodiment shows an eleven electrode probe with 11 wired connections cable of generating 8 independent CSA measurements. In reality many more CSAs can be measured depending on the size of the sphincter region under investigation and the spacing required between measurements.

The sketch demonstrates one embodiment with 8 impedance planimetric recording of CSAs inside the balloon along with pressure recordings. This embodiment has a rather long balloon so the catheter can slide inside the balloon in order to obtain a profile of the geometry of the sphincter. This is demonstrated in FIG. 4 where the bag-like balloon maintains its approximate position while the position of the electrodes can be changed either left or right of their original position in a longitudinal direction with reference to the lumen. The diagram shows the probe with the activation and sensing system required to display information from the collected data.

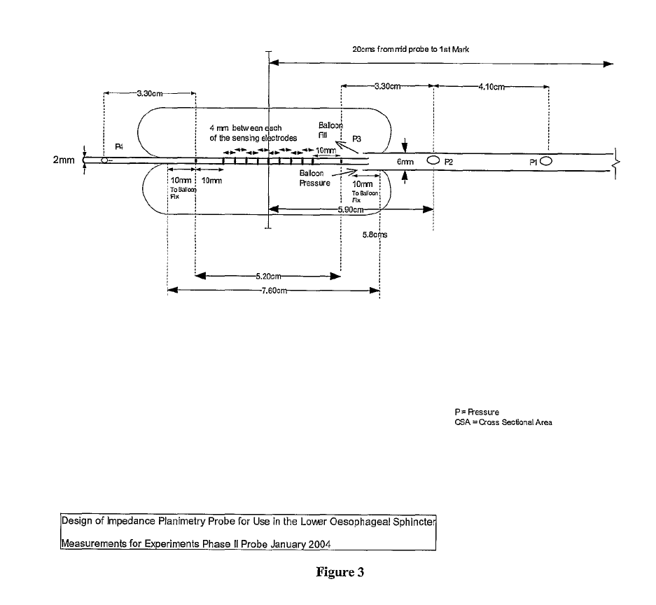

FIG. 3 shows an apparatus being constructed in such a manner that the measurement end of the apparatus has a very small diameter allowing the distension of sphincteric region such as the oesophagogastric junction to be distended from as close to the resting state as possible, the resting state being when the musculature of the region forms a closed area of toned muscle. In the embodiment shown the luminal body of the catheter is approximately 6 mm in diameter and the distal measurement section of the catheter is approximately 2 mm in diameter.

In FIG. 7, the apparatus shown is a longitudinal catheter placed in a sphincter or narrowing region of the body. However, the apparatus may be used in animals, plants, as well as non-biological systems such as process systems where luminal channels exist that may have regions prone to narrowing and where the measurement of such narrowing may be important within the context of this section of the system, machine or process. Furthermore, the apparatus may be used in any hollow system of the body of the person or the animal, either in vivo or in vitro where there is a sphincter or narrowing region which may need to be investigated from a biomechanical point of view, non-limiting examples of such regions being: the lower oesophageal sphincter, the pyloric sphincter and the anal sphincter in the gastrointestinal tract, the urethral sphincter a part of the urogenital system, or obstruction in the cardiovascular system including the heart or other systems.

FIG. 8 shows an example of distensions plots. In this case the balloon can be inflated and deflated in a controlled manner either manually but preferably with a pump that can be used manually or controlled by a PC. As illustrated in the figure the infusion rate can be varied and the distension repeated during different drug treatments. The distension is done by means of a fluid, preferably a liquid, and more preferred a saline solution introduced to the balloon through a channel provided in the catheter. The fluid is pumped to the balloon from an exterior reservoir such as a sterile bag containing the fluid, the pumping being provided by, as example, a pump with rollers exerting a pumping action on a hose or a syringe driver pump and the fluid is pumped from the reservoir to the proximal end of the catheter.

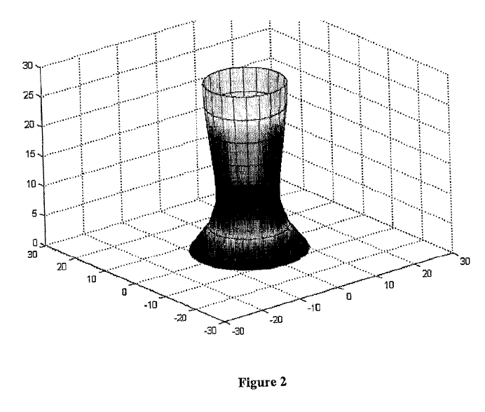

The pressure of the fluid is monitored, possibly by a pressure gauge inside the balloon. Alternatively to measuring the pressure, the volume of the fluid being pumped may be monitored, either when being pumped to the balloon or after having been pumped to the balloon. The inflation of the balloon constitutes a mechanical stimulus of the sphincter or of a hollow organ restriction in the area under investigation. This is demonstrated in FIG. 2 where the surface geometry of the sphincter is reconstructed using 8 CSAs and shown on a 3-dimensional diagram.

The catheter is provided with a number of channels running inside the catheter. Some of the channels are intended for passing stimulating means or measuring means from the proximal end of the catheter to a more distant end of the catheter, either at a position before the balloon, or at a position inside the balloon or a position after the balloon toward the distal end of the catheter.

With reference to the figures, it is important to notice that the catheter and the balloon only form part of the apparatus according to the invention, i.e. the apparatus does not consist of the catheter and the balloon, but the apparatus comprises the catheter and the balloon. Other parts of the apparatus may comprise any exterior equipment for generating the stimuli and any additional equipment for recording data, possibly provided by the gauges or other recording means attached to, connected to or in any other way assigned to the catheter and the balloon.

Data collected on the multiple CSAs, volume and pressure inside the balloon could be used to calculate wall tension, to derive force data such as total force and passive force. Strain may be calculated from data on volume, diameter and CSA.

Geometric data may be gathered using radiological or non-radiological imaging equipment such as X-ray, MRI, and Ultrasound. Miniature ultrasound probes may be used inside the balloon of the probe.

Pharmaceutical agents for muscle relaxation or muscle stimulation may be used. Information may be gathered on layer thickness and calculations may be made of longitudinal forces for data gathered.

Miniature ultrasound transducers may be built-in to the probe assembly to image local tissue.

Electrodes inside the probe may be ring shaped or pointed in shape or indeed any other shape.

Data from electrodes inside the probe balloon may be collected for common wire arrangements which may allow switching between wires to optimise usage. Multiplexing of the signals may also be used to reduce number of connections and amount of hardware for signal processing.

Electrodes may be constructed from thin, flexible, prefabricated circuits with conductive tracks which are then affixed to catheter.

Data gathered may be used to construct the 3D geometry of the narrowing region under investigation.

The OGJ may be defined by the measurements made with this probe.

In one embodiment of the invention, the most narrow location in the bodily sphincter, the length of the bodily sphincter and the volume of the bodily sphincter and the lumen are determined at various loading levels by means of curve-fitting of the geometric and biomechanical data and curve analysis in terms of differentiation or other relevant mathematical functions, such as to define local minimum or maximum.

For example when the CSA is measured at multiple sites around and inside the sphincter, the radius and circumference can be computed under assumption of circular geometry. If the radius or circumference curves as function of the length (axial direction) and at one or several pressure levels are differentiated, then the sphincter length can be defined in various ways, for example by using the inversions points (local maximum or minimum on the differentiated curve). Another method to determine sphincter length is to define a certain change in CSA, circumference or radius from a baseline level. Other definitions can be made using similar principles.

### EXAMPLE OF A POSSIBLE APPARATUS AND EXPERIMENT

An eleven-electrode impedance measuring system located inside a balloon on a 120-cm-long probe was used for measurements of luminal cross-sectional area (CSA) in the LOS similar to the embodiment shown in FIG. 3. The CSA can be measured according to Ohm's law from measurements of the impedance of the fluid inside the balloon. The attached balloon could be 80 mm long but fixed to the catheter along a section that is 40 mm long and encompassing all the electrodes and was made of 50-μm-thick non-conducting polyurethane. The balloon was connected via an infusion channel (2.5 mm in diameter) to a syringe that could be mounted into a syringe driver and pushed or withdrawn to alter the balloon volume. Thus, the balloon could be inflated with an electrically conducting fluid (0.00225% NaCl) to a maximum CSA of approximately 600 mm² (diameter 30 mm) without stretching the balloon wall. Thus, reliable measurements could be carried out in the physiological range without stretching the balloon wall. The probe contained three channels for pressure measurement. One side hole was located inside the balloon between the detection electrodes, while the other two side holes were located 4 cm proximal and distal to the balloon, respectively.

A group of controls and patients with pH positive GORD are fasted for 8 hours before the procedure.

The probe was passed into the oesophagus via the nostrils after calibration of the impedance planimeter. The balloon was positioned using the pressure channels for guidance into the LOS.

To fine tune the position of the probe and to confirm that the balloon was definitely in the sphincter, 20 ml was filled into the balloon. The tracings were observed and because of the oversized balloon the catheter with the electrodes on it could be moved slightly up or down until the least distended CSAs i.e. those at the most closed or narrow point of the sphincter were detected by the central CSA measurement electrodes.

The probe was then taped to the nose. The subjects were asked to lie flat in supine position on the bed at the same level as the pressure transducer and to relax for 30 minutes. Volume-controlled distensions were performed at 40 ml per minute and then the solution was withdrawn at the end of each distension. The balloon was emptied for several minutes between the distensions. During the distension, the subjects were asked to report any visceral perception on a visual analogue scale meter. Twenty minutes after finishing the first distension series, another similar series were done in order to evaluate the reproducibility. Twenty minutes after finishing the reproducibility test, a third distension series was done during administration of the anti-muscarinic agent butylscopolamine (20 mg) in order to relax the smooth muscle. The total butylscopolamine dose (20-120 mg) was guided by the degree of abolishment of contractions and by the development of classic anticholinergic side effects.

Before the test started the subjects were trained how to use the VAS meter. First, they were asked to report the sensation to somatic stimuli (increasing pressure applied to the right forearm) and second, they scored symptoms during a few balloon distensions. The intensity of the non-painful sensations were scored on a 1-5 scale, where 1=vague perception of mild sensation; 2=definite perception of mild sensation; 3=vague perception of moderate sensation; 4=definite perception of moderate perception and 5=discomfort. The subjects had the ability to score half units on the scale.

The CSA was estimated at several locations along the sphincter during stepped and ramped distensions. If sphincter relaxations were present, the mean CSA and pressure were used for further analysis. The balloon distension induced relaxation of the LOS.

Positioning of the balloon was vital to the experiment.

The contraction frequency proximal and distal to the balloon was scored. The circumferential wall tension was calculated according to the law of Laplace for cylindrical structures as

T=ΔPr

where T is the circumferential wall tension (kPa m), r is the balloon radius, and ΔP is the transmural pressure. Other suitable or possible ways of computing the tension and stress such as Cauchy-stress and Kirchhoff-stress may be applied under different geometric assumptions and depending on the design and function of the apparatus. The balloon pressure at a volume of 3 ml during the administration of butylscopolamine was assumed equal to the resting pressure of oesophagus (i.e. the intra-abdominal pressure). Hence, the transmural pressure during distension was considered to be equal to the difference between the balloon pressure and the resting pressure. The total tension (Tₜₒₜₐₗ) during distension (due to both active and passive tissue properties) was determined from the distension test without the administration of butylscopolamine. The passive tension (Tₚₐₛₛᵢᵥₑ), that only results from passive components such as the extracellular collagen was obtained from the test with butylscopolamine. The active tension (T_{active}) contributed by smooth muscle activity was computed using the equation:

Tₜₒₜₐₗ=T_{active}+Tₚₐₛₛᵢᵥₑ

Other suitable or possible ways of computing the relationship between these tensions may be applied under different geometric assumptions and depending on the design and function of the apparatus.

The circumferential strain is the fractional change in radius computed as

$$ɛ = \frac{r - {r}_{0}}{{r}_{0}}$$

where r is the radius at a given distension and r₀ is the reference radius at a wall tension of 0.002 kPa m under the assumption that the geometry was circular. At the reference tension, it was easy to determine r₀ graphically for the different subjects. Other suitable or possible ways of computing the strain such as Cauchy-strain and Green-strain may be applied under different geometric assumptions and depending on the design and function of the apparatus.

Stepwise inflation of the balloon resulted in a consistent mechanical response. Butylscopolamine clearly inhibited the contractile activity.

An example of data obtained during a single manually controlled distension of the sphincter is shown in FIG. 8. The balloon pressure curve shows how pressure builds up in the balloon as a volume of saline is filled over a period of about 2 minutes into the balloon at a steady rate. In this example the electrode pairs are 4 mm apart and the distal pair are measuring distal CSA. As this electrode pair is in the stomach the saline takes the path of least resistance and as expected the balloon bulges into the stomach giving a large CSA even at low pressures. Then as the volume continues to fill, the proximal and mid CSAs get larger and the balloon pressure increases. The reverse happens when the saline is removed from the balloon by reversing the syringe.

Calculations of flow through the LOS during swallowing can also be made when the probe is in situ in the sphincter. The patient performs both water and air swallows and the CSA and pressure readings are recorded for the relaxation of the LOS as a result of the swallows. Using Newton's law of motion applied to force, rates for air and water in both the control and patient groups can be estimated using the following equation;

$$Q = dp \times \frac{{D}^{4}}{CVL}$$

Q=flow rate, dP=Pressure Difference, D=Diameter, C=Constant, V=Viscosity, L=length

Other suitable or possible ways of computing the flow rate may be applied under different geometric assumptions and depending on the design and function of the apparatus.

1-40. (canceled)

41. Apparatus for measuring sphincter or narrowing region geometry, competence, and distensibility, when a sphincter or narrowing region of a human body, animals, plants and non-biological systems is being subjected to an artificially applied mechanical stimulus, said apparatus comprising:
a catheter being provided with an inflatable balloon situated between a proximal end and a distal end of the catheter,
a means for passing an inflating substance from the proximal end to the balloon,
a means for measuring at least one of the volume of the balloon, the transverse cross-sectional area of the balloon, the diameter of the balloon, the tension in the balloon, the strain in the balloon, the pressure of a fluid inside the balloon, a force induced by the balloon onto the sphincter narrowing region, and a deformation induced by the balloon on the sphincter or narrowing region,
a means for imaging of a physical state of the walls of the sphincter or other narrowing region, and
a means for deriving data from the measuring means on change of geometry and the force-deformation relationship establishing change of geometry, lumen dynamics or tissue properties of the sphincter or narrowing region caused by a disease or a treatment.

42. Apparatus as claimed in claim 41 in which the measuring means is selected from one or more of the following:
a strain gauge, a pressure gauge, a temperature gauge, a piezo-electric gauge, electrodes, a pH-recording means, an electromyographic (EMG) recording means, an ultrasonic measuring means, a visual recording means, a scanning means, a MR scanning means, a CT scanning means and a means for recording flow of fluid.

43. Apparatus as claimed in claim 41 in which a number of the measuring means are located in the balloon.

44. Apparatus as claimed in claim 43 in which the measuring means located in the balloon are selected from one or more of the following:
a pressure gauge, a temperature gauge, an ultrasonic measuring means, a visual recording means, a MR scanning means, a CT scanning means, an electromyographic (EMG) recording means and a means for recording flow of fluid.

45. Apparatus as claimed in claim 41 in which a number of the measuring means are located outside the balloon.

46. Apparatus as claimed in claim 45 in which the measuring means located outside the balloon are selected from one or more of the following:
an ultrasonic measuring means, a visual recording means, a scanning means, a MR scanning means, a CT scanning means, an electromyographic (EMG) recording means and a means for recording flow of fluid.

47. Apparatus as claimed in claim 41 in which a means for recording and controlling a temperature in the balloon is provided.

48. Apparatus as claimed in claim 47 in which the means for recording and controlling a temperature in the balloon is located adjacent the balloon boundaries adapted for abutting a wall of the sphincter or narrowing region.

49. Apparatus as claimed in claim 41 in which the diameter of an end of the apparatus adjacent the measuring means is very small for allowing the sphincter or narrowing region to be distended from as close to the resting state as possible, the resting state being when the musculature of the sphincter or narrowing region forms a closed area of toned muscle.

50. Apparatus as claimed in claim 41 in which an outer surface of the balloon is one of roughened, serrated, made uneven by a manufacturing or treatment process for restricting longitudinal movement of the balloon in the sphincter or narrowing region.

51. Apparatus as claimed in claim 41 in which a second balloon is placed more proximally or more distally than the said balloon for facilitating stimulation to the sphincter or narrowing region or other part of a lumen containing the sphincter or narrowing region so that the sphincter or narrowing region may respond by one of relaxing, contracting or behaving in a way that can be detected by the measuring means.

52. Apparatus as claimed in claim 41 in which a plurality of ring electrodes or point electrodes are provided in combination with a multiplexing system or a common wired electrode system thereby minimising the number of electrodes or connections for driving output of data therefrom.

53. Use of the apparatus as claimed in claim 41 for determining at least one of the parameters of geometry competence and distensibility of a sphincter or a narrowing region of a bodily hollow system.

54. A method for investigating multiple simultaneous measurements in sphincters and other narrowing regions on the human body, animals, plants and non-biological systems so as to determine geometry, competence, surface geometry and distensibility, the method comprising:
inflating a balloon to an extent for the balloon to be fixed in relation to the hollow system,
introducing a mechanical stimulus by the inflated balloon to the narrowing region, said mechanical stimulus being introduced between the exteriorly accessible opening of the hollow system and the distal end of the catheter through channels inside the catheter,
measuring a change of a physical property of the balloon during inflation of the balloon, said physical property being one of a dimensional state of the balloon, a force induced to the balloon, a mutual state between the balloon and the wall of the hollow system,
deriving, from the measurements, data on change of geometry and the force-deformation relationship in and the sphincter or narrowing region of the hollow system, and
establishing, from the deriving of data on change of geometry and the force-deformation relationship, change of geometry, lumen dynamics and tissue properties caused by a disease or a treatment.

55. A method as claimed in claim 54 in which the method is performed anywhere in sphincters, natural narrowings or unnatural narrowings such as obstruction associated with disease state or as a result of treatment or trauma in one of the following bodily hollow systems: the digestive system including the stomach, the urogenital tract including the bladder, the cardiovascular system including the heart, the ear canal including the eustachian canal and the posterior nares.

56. A method as claimed in claim 54 in which a local curvature is derived in various directions from the multiple measurements of geometric and force data.

57. A method as claimed in claim 54 in which the deriving of the geometric measures such as pressure-diameter diagrams, multiple cross-sectional areas used to make calculations along the length and breadth of the balloon, the 3D-geometry of the balloon and computing data related to total force tissue properties and computing data related to passive force tissue properties, and where data related to active force tissue properties is computed based on the data related to the total force tissue properties and to the passive force tissue properties, and preferably, computing of the geometric data related to the passive force tissue properties is performed during muscle relaxation such as by muscle relaxing drugs, and advantageously, computing of the geometric data related to the total force tissue properties is performed during muscle stimulation such as by muscle stimulating drugs, and preferably, computing of the geometric data related to the total force tissue properties is performed during no muscle stimulation by drugs, and advantageously, computing of the geometric data related to the active force tissue properties is performed from values measured during introduction of a balloon into a hollow system showing short-lasting muscle contractions, and preferably, computing of the data related to the active force tissue properties is performed from values measured during the introduction of a balloon into a hollow system showing long-lasting muscle contractions, and advantageously, computing of the geometric data related to the active force tissue properties is intermediate values computed from values measured during a balloon being introduced into a hollow system showing any type of muscle contractions.

58. A method as claimed in claim 54 in which the lumen dynamics are changed by way of inflating the balloon, thereby introducing a change in pressure inside the balloon, said pressure being used for computing the circumferential tension applied by the balloon to the internal surface of the wall of the hollow system, and said change in pressure being measured as the measure of the magnitude of mechanical stimulus, or alternatively, the lumen dynamics are changed by way of inflating the balloon thereby inducing a change of volume of the balloon, said volume being used for computing the circumferential strain applied by the balloon to the internal surface of the wall of the hollow system, and said change in volume being measured as the measure of the magnitude of mechanical stimulus, or alternatively, the lumen dynamics are changed by way of inflating the balloon thereby inducing a change of a diameter of the balloon, said diameter being used for computing of the circumferential strain applied by the balloon to the internal surface of the wall of the hollow system, and said change of diameter being measured as the measure of the magnitude of mechanical stimulus, or alternatively, the lumen dynamics are changed by way of inflating the balloon thereby introducing a change in cross-sectional area of the balloon, said area being used for computing of the circumferential strain applied by the balloon to the internal surface of the wall of the hollow system, and said change in cross-sectional area being measured as the measure of the magnitude of mechanical stimulus, or alternatively, the lumen dynamics are changed by way of inflating the balloon thereby introducing a change in circumferential tension of balloon, said tension being used for computing of the circumferential tension applied by the balloon to the internal surface of the wall of the hollow system, and said change in tension being measured as the measure of the magnitude of mechanical stimulus, or alternatively, the lumen dynamics are changed by way of inflating the balloon thereby introducing a change in local curvature in various directions of the balloon, said curvature being used for computing of the circumferential tension applied by the balloon to the internal surface of the wall of the hollow system, and said change in tension being measured as the measure of the magnitude of mechanical stimulus, or alternatively, the lumen dynamics are changed by way of inflating the balloon thereby introducing a change of a number of dimensions of the balloon, said inflation inducing a circumferential strain applied by the balloon to the internal surface of the wall of the hollow system, and said change of strain being measured as the measure of the magnitude of mechanical stimulus.

59. A method as claimed in claim 54 in which the location and length of the sphincter or narrowing region is exactly identified by having measurements of cross-sectional area on at least one side, possibly on both sides, and on the inside the sphincter or other narrowing region, and preferably, the geometry of the sphincter or other narrowing region is obtained applying a pull-through technique, said technique being capable of measuring lumen diameters, a single or multiple cross-sectional areas along the length of the balloon, circumferences, the contour, the three-dimensional geometry or the wall thickness of the region of interest with or without the balloon pressure, and advantageously, the pull-through is performed by inflating a balloon, said balloon being fixed to the wall and the catheter being moved stepwise or continuously inside the balloon placed in the region of interest, and preferably, an ultrasonic probe is inserted into the balloon and that cross-sectional areas are measured by radial scanning by the probe, said probe being free to move within the measurement range of the inflated balloon, and preferably, the probe is constructed with ultrasound crystals in situ for the imaging of luminal walls for the purpose of determining cross-sectional areas and other geometry in sphincteric and other narrowing regions of the hollow organs.

60. A method as claimed in claim 54 in which measurement of multiple cross-sectional areas and other data such as pressure and distance is used in relation to either a stepped or pull-through technique in a bodily sphincter or other narrowing region and in the near lumen proximal and distal to the sphincter or narrowing region under investigation to calculate or interpolate longitudinal forces in the sphincter, near the sphincter or in or near the narrowing region, and advantageously, the most narrow location in the bodily sphincter or other narrowing region is determined at various loading levels by means of curve-fitting and curve analysis in terms of differentiation or other relevant mathematical functions, such as to define local minimum or maximum, and advantageously, the lengths of the bodily sphincter or other narrowing region is determined at various loading levels by means of curve-fitting of the geometric data and applying differentiation or other mathematical functions, such as to define local minima and maxima, and preferably, the volume of the bodily sphincter or other narrowing region and the volume of the lumen can be determined at various loadings levels by applying curve-fitting and mathematical functions, and advantageously, measurement of the narrowest cross-sectional area of the bodily sphincter or other narrowing region can be determined at various loading levels by review of a multiple cross-sectional areas being simultaneously measured.
